(19) United States
(12) Patent Application Publication
(10) Pub. No.: US 2015/0133907 A1
(43) Pub. Date: May 14, 2015

(54) Integrated Suture and Cauterization

(71) Applicants: Paul H. Chen, Rancho Santa Fe, CA (US); Robert Hotto, Carlsbad, CA (US)

(72) Inventors: Paul H. Chen, Rancho Santa Fe, CA (US); Robert Hotto, Carlsbad, CA (US)

(21) Appl. No.: 14/603,155

(22) Filed: Jan. 22, 2015

Related U.S. Application Data
(63) Continuation-in-part of application No. 13/215,407, filed on Aug. 23, 2011.

(51) Int. Cl. A61B 18/10; A61B 18/08; A61B 18/14

(52) U.S. Cl. CPC A61B 18/10; A61B 18/1477; A61B 18/08; A61B 2018/00595
USPC 606/30

(57) ABSTRACT

A system and method for inhibiting bleeding during surgical wound closure is provided. As the surgical wound created as part of surgery is closed by a suture and needle, energy is applied to the needle causing bleeding tissue to cauterize thus inhibiting at least some of the bleeding that invariably accompanies surgery. The surgical needle is held by a needle holder or other device and that device holding the needle is connected to an energy source that can be controlled by the surgeon. As energy from the energy source is applied to the needle holder through a conductive line, it is conveyed to the surgical needle through the needle holder and the energy emanating from the needle cauterizes tissue and controls bleeding as the wound is sutured closed. Bleeding from the needle tract created by the puncture of the tissue by the sharp point of the needle and passage of the needle body into the tissue that would otherwise occur as a necessary part of suture is in particular inhibited. Use of the system and method in blepharoplasty is described.

### RELATED APPLICATIONS

This application is a continuation-in-part of U.S. patent application Ser. No. 13/215,407 filed Aug. 23, 2011, which application is hereby incorporated by reference.

### TECHNICAL FIELD

This invention relates to suturing and cauterizing devices and systems employed in surgery and medicine.

### BACKGROUND

Bleeding is part of many surgical procedures, including, for example, neurological, skin, cardiothoracic, vascular, and abdominal surgery. Surgical bodily repair typically requires bodily tissue incision before targeted areas are reached. Bleeding inevitably ensues from the incision itself and the concomitant closure of the surgical wound with staples or suture. Bleeding adds a risk quotient to surgery and presents in a variety of modes with variable predictability. Not only is bleeding an inherent artifact of tissue incision, suturing itself precipitates bleeding as a needle is drawn through the tissue as the surgical opening is closed leaving tracts that bleed along with bruising arising from undesired blood accumulation along the tract and associated impact on healing. Consequently, bleeding is an inherent part of surgery and its control is an important consideration for the surgeon.

A variety of tactical procedures and instruments have, therefore, been devised to reduce unwanted bleeding during surgical procedures. Those prior procedures and instruments have, however, typically contemplated bleeding control as a discrete or separate step in surgical procedure. Separate cauterization of bleeding takes additional time and risks cutting the suture.

In other instances, specialized tools such as, for example, cauterizing staplers have been employed to minimize bleeding during closing. Surgical staplers are, however, limited. They are more cumbersome than sutures and cannot be used in many situations such as, for example, on small structures and in confined areas such as near the eyes, for example. In addition, surgical staples are less secure than sutures and do not provide a continuous sealed tract as can sutures. Further, staplers can leave a more prominent scar than closure with suture.

Sutures on the other hand pass through tissue and consequently leave tracts about which there is peripheral bruising and along which there is bleeding both of which can impact healing progress and the visual appearance of the area once healed.

Consequently, what is needed is a system for wound closure and tract cauterization that can improve surgical technique and efficiency yet can be employed in a variety of fields and at various scales with disposable or reuseable tools. Consequently, the present invention provides instruments and procedures to suppress bleeding through application of energy to the surgical needle and, in alternative embodiments, to the suture line.

### SUMMARY OF INVENTION

A system and method for inhibiting bleeding during surgical wound closure is provided. As the surgical wound created as part of surgery is closed by a suture and needle, energy is applied to the needle causing bleeding tissue to cauterize thus inhibiting at least some of the bleeding that invariably accompanies surgery. The surgical needle is held by a needle holder or other device and that device holding the needle is connected to an energy source that can be controlled by the surgeon. As energy from the energy source is applied to the needle holder, it is conveyed to the surgical needle through the needle holder and the energy emanating from the needle cauterizes tissue and controls bleeding as the wound is sutured closed. Bleeding from the needle tract created by the puncture of the tissue by the sharp point of the needle and passage of the needle body into the tissue that would otherwise occur as a necessary part of suture is inhibited. Various powers may be applied with the present system and method varying from sensitive applications such as blepharoplasty to more robust applications with heavier tissue applications.

### BRIEF DESCRIPTION OF FIGURES

FIG. 1 depicts an embodiment of the present invention that provides energy to a conductive surgical needle.

FIG. 2 depicts needle holder as connected through a plug to a conductive line from an energy source.

FIG. 3 depicts the area within circle 3 of FIG. 2 and, in particular, jaws and the respective faces of an exemplar needle holder in accordance with an embodiment of the present invention.

FIG. 4 depicts an exemplar surgical needle.

FIG. 5 depicts an example alternative configuration for a needle holder as may be employed in various embodiments of the present invention.

FIG. 6 depicts use of an exemplar system in accordance with an embodiment to close a surgical wound with what is sometimes called a running technique.

FIG. 7 illustrates use of an exemplar embodiment to assist in bleeding control during closure of a surgical wound.

FIG. 8 depicts closure of a surgical wound after upper eyelid blepharoplasty.

### DETAILED DESCRIPTION OF PREFERRED EMBODIMENTS OF THE PRESENT INVENTION

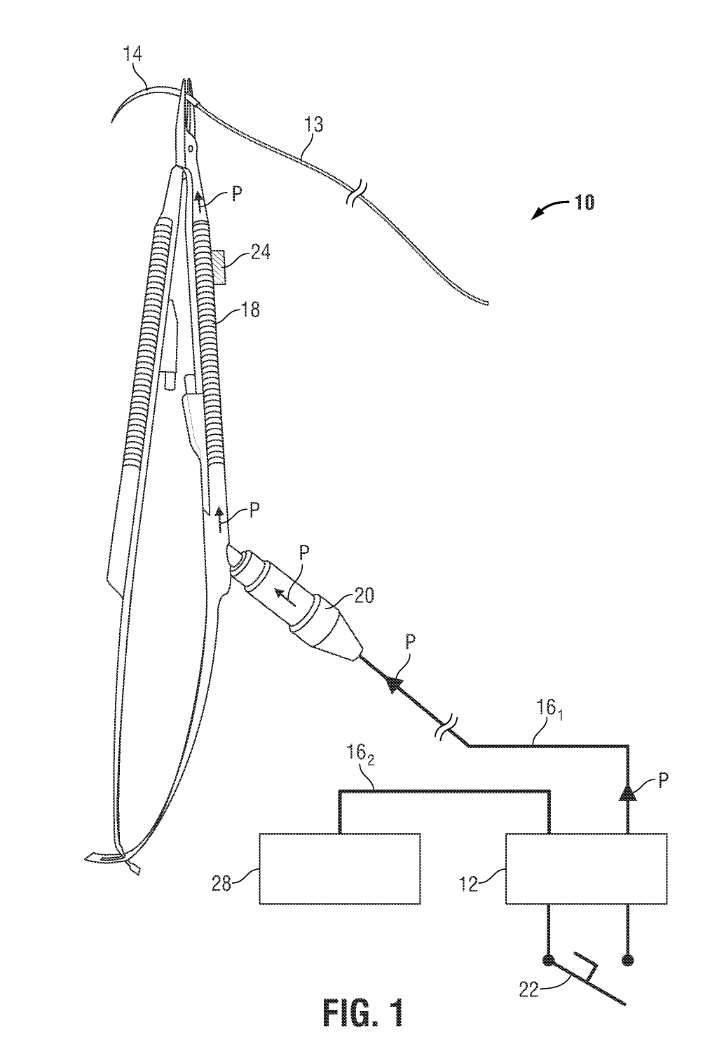

FIG. 1 depicts an embodiment of the present invention that provides energy to a conductive surgical needle 14 to precipitate tissue cauterization. System 10 depicted in FIG. 1 is particularly useful in controlling bleeding arising from the suture tracts that inevitably are created during wound closure as will be further described below. As shown in FIG. 1, energy source 12 is connected to conductive line 16₁ which is connected to needle holder 18 through plug 20 and thus, when needle 14 is grasped by needle holder 18, an energy conduction path P from energy source 12 along conductive path 16₁ through plug or connection 20 through needle holder 18 to surgical needle 14 is created along which energy from energy source 12 travels to needle 14 from which energy emanates when energy source 12 is enabled. Conductive line 16₁ is preferably insulated to prevent unintended conduction to health care providers or other surgical equipment. A variety of methods and structures can be used to connect needle holder 18 to conductive line 16₁ and a plug adapted to convey RF energy is shown although a variety of connectors and ways to connect conductive line 16₁ to needle holder 18 are known and available and may include, for example, a variety of fittings and attachments permanent and detachable adapted for conveyance of energy conveyed from energy source 12 through conductive line 16₁. Whatever is used to connect conductive line 16₁ to needle holder 18, maintaining a low impedance path is preferred particularly when energy from energy source 12 is radio frequency (RF) energy.

Preferably, energy source 12 is an RF energy source. Common types readily available to the practitioner include electrosurgical generators such as those provided, for example, by Conmed, Ellman or Valley Lab. Those and other cautery units that generate controllable RF with various waveforms available for cautery are commonly available and known to the surgical practitioner and may be employed as energy source 12 as may electrical supplies that provide lower frequency electrical energy so long as cauterization can result from the application of the energy along path P from energy source 12 to needle 14. Further, as those of skill will appreciate, when cauterizing needle tracts such as those shown in later figures and identified by reference 46, it is preferred to employ energy from energy source 12 devised for cauterization rather than cutting tissue. Such choices of energy function typically based on waveform patterns are known to be available from commercially available cautery units. In any case, as the experienced practitioner will appreciate, care should be taken to moderate the power applied to avoid deleterious tissue impact.

Energy source 12 produces energy whose energy can be conveyed along conduction path P. Energy source 12 is preferably a monopolar RF source to provide energy to surgical fields without requiring a conductive return plate in contact with the patient. Optional conductive line 16₂ is shown in FIG. 1 attached to optional conductive contact or plate 28 that is placed in contact with a patient when a separate and literal return path is required for energy conveyance through needle 14. Energy source 12 is preferably surgeon or practitioner-controlled and a variety of control apparatus are known in the art such as exemplar foot switch 22 or a thumb controlled switch 24 typically sited on the shaft of needle holder 18 to control energy source 12 as deemed appropriate by the practitioner. Selective enablement of energy source 12 provides control over a cauterization field that can be contained to the suture tract at lower powers and durations or extend into the wound area itself at higher powers and even in some cases provide cauterization to the wound.

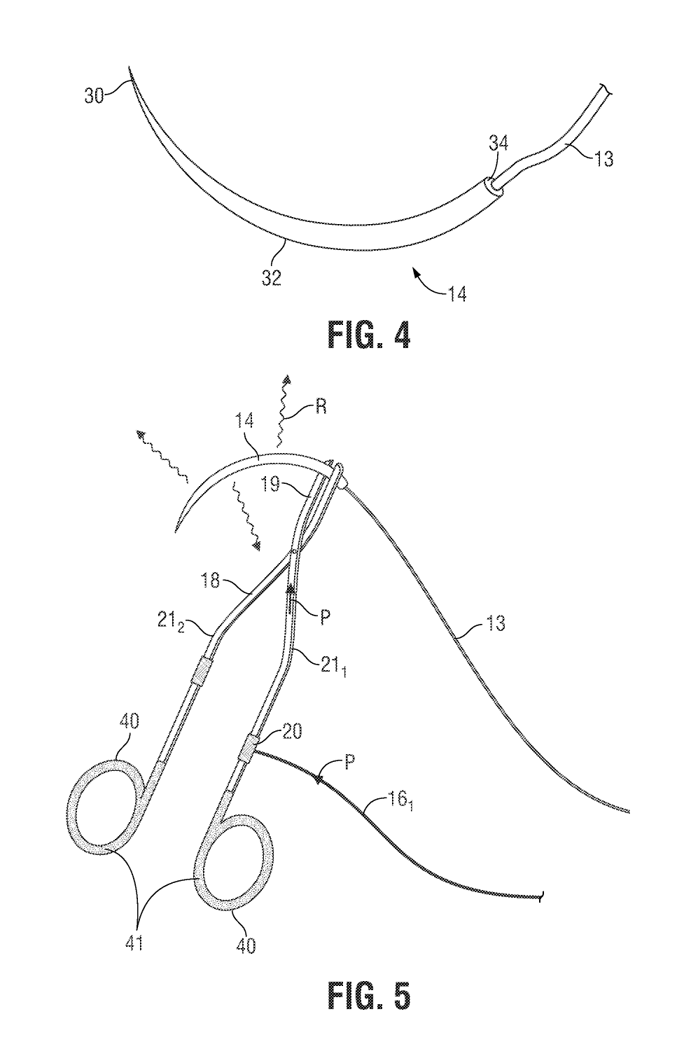

Needle holder 18 is depicted as a needle holder grasping needle 14. As shown, suture line 13 is attached to surgical needle 14 at its swag as will be shown in greater detail in later FIG. 4. Needle holder 18 is, as those of skill will recognize, but one of many different types of structures useful in providing hand control over manipulation of needle 14. Other labels applied to such devices that may be employed for needle manipulation in surgery include, just as examples, “clamp” or “forceps” but any device that allows controllable manipulation of needle 14 should be deemed for purposes of this disclosure a needle holder 18.

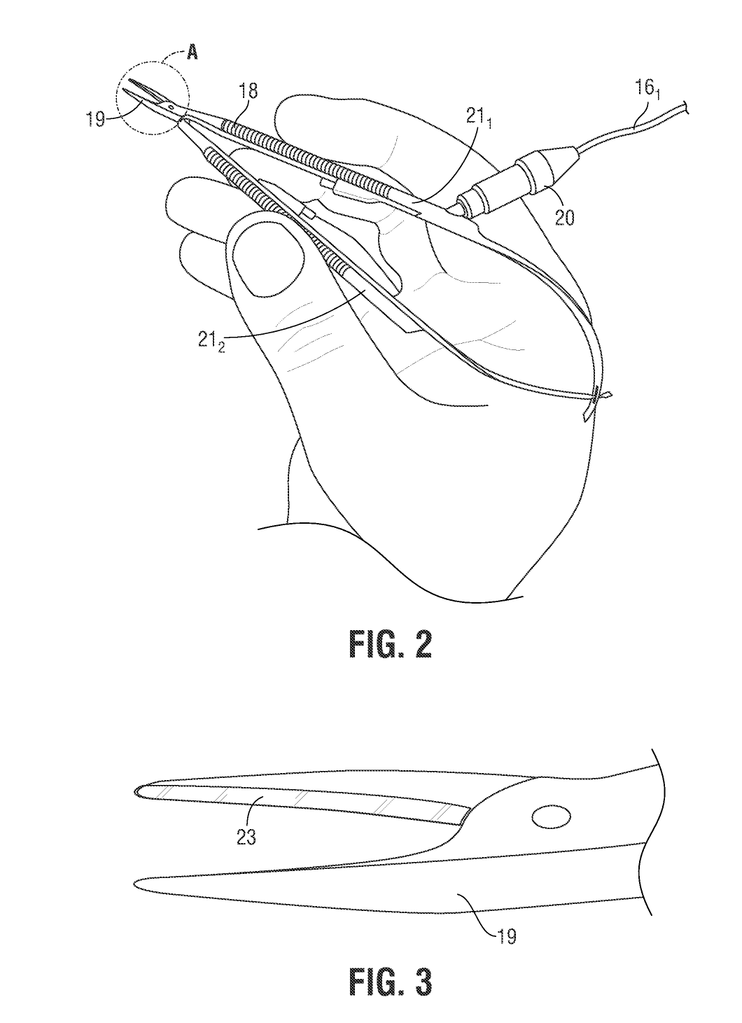

FIG. 2 depicts needle holder 18 as connected through plug 20 to conductive line 16₁ from energy source 12. Needle holder 18 is shown in a practitioner's hand but it should be appreciated that various embodiments of the present invention may be employed to advantage with a robot as shown in incorporated application U.S. patent application Ser. No. 13/215,407.

Needle holder 18 is comprised from material conductive to the energy applied and in the depiction of FIG. 2, needle holder 18 is shown with arms 21₁ and 21₂ which are, in this depiction, insulated to reduce energy transmission into the practitioner's hands. Gloves are typically employed during surgery as gloved hand manipulation is greatly preferred, as those in the art will recognize, and gloves will offer substantial insulative function but even so, insulation of particularly handled parts of needle holder 18 are preferred particularly when using RF as RF can pass through some otherwise insulative materials.

FIG. 3 illustrates in greater detail the area within circle A of FIG. 2. As indicated, jaws 19 of needle holder 18 are conductive on at least their respective faces 23. The connection of energy source 12 (not shown in this Fig.) to needle holder 18 allows the supplied energy to be conveyed along path P as shown in FIG. 1 to surgical needle 14 held in conductive-faced jaws 19 of the needle holder 18.

FIG. 4 depicts an exemplar surgical needle 14. As those of skill will understand after appreciating this disclosure, an uninsulated surgical needle is indicated for use with the present invention so that the body 32 of needle 14 may be readily grasped with needle holder 18 during suturing. Thus by being preferably uninsulated, surgical needle 14 provides grasping flexibility which would not be available with a needle that had only part of its conductive surface closed. Flexible access to the needle is an advantage during surgery and using an unisulated needle provides that flexibility in the context of the present invention. As shown in FIG. 4, needle 14 exhibits sharp point 30 for initial penetration of tissue during suturing. Body 32 is conductive so that the conductive jaws of needle holder 18 may provide a conductive grasp of needle 14 along body 32. As shown, swag end 34 is configured to allow ready attachment of suture line 13.

FIG. 5 illustrates a forceps scissor style needle holder 18 grasping surgical needle 14. Although the surgeon typically uses gloves, needle holder/clamp 18 preferably has nonconductive surfaces along arms 21₁ and 21₂ to allow the practitioner convenient handling. For example, non-conductive surfaces 41 are shown on needle holder/clamp 18 depicted in FIG. 2 on finger loops 40 of a forceps style needle holder 18 used to hold needle 14. Insulation may be employed on arms 21₁ and 21₂ of needle holder 18 or, alternatively, the arms themselves may be comprised from non-conductive material so long as a conduction path P is created from energy supply 12 along conductive line 161 to needle holder 18 and then to jaws 19. Non-limiting examples of an insulative covering for needle holder 18 may include rubber or plastics or insulative paint and coatings of a variety of types known in the arts. As represented in FIG. 5, when surgical needle 14 is grasped by jaws 19 of needle holder 18 and energy source 12 is enabled, energy will emanate from surgical needle 14 as represented by reference “R”.

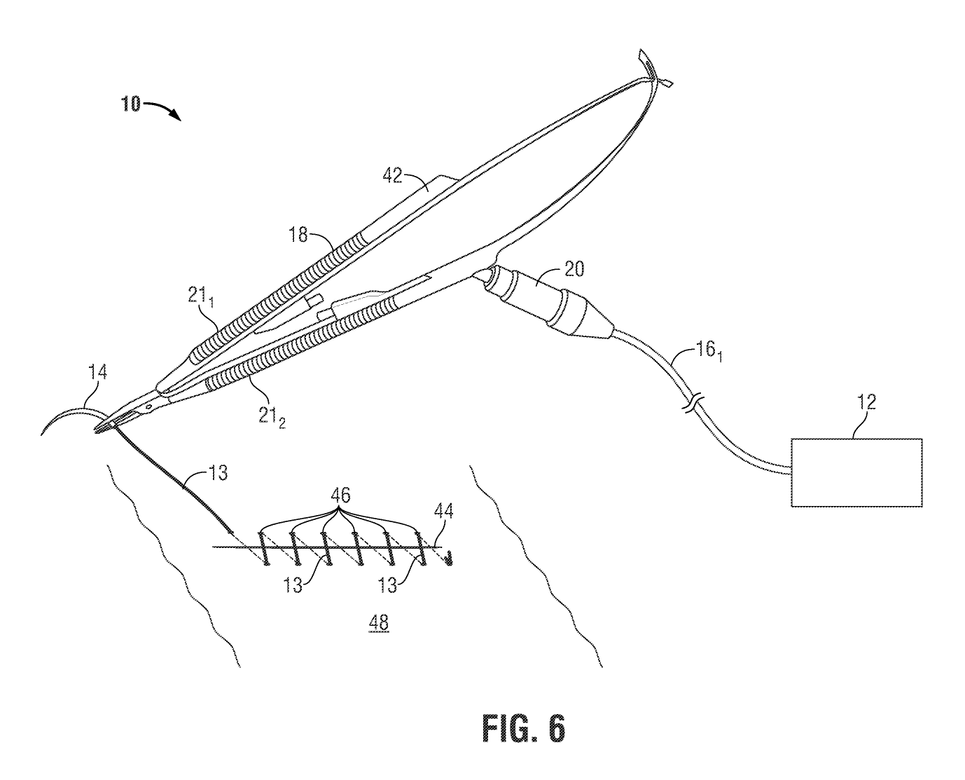

FIG. 6 depicts a preferred exemplar use of system 10 in closure of surgical wound 44 with what is sometimes called a running technique. Needle 14 is inserted in tissue field 48 under the derma including whatever associated subdermal tissue is indicated by the dimensions, location and type of tissue to be closed and RF energy is applied for between 1 to 10 seconds resulting in a cauterization zone of approximately from 0.1 mm to 1 mm thus providing cauterization and bleeding control particularly of the needle tracts as depicted by references 46 in FIG. 6. Needle 14 is then pulled up through the other side of wound 44 creating another tract 46 similarly cauterized by the applied RF energy as wound 44 is incrementally closed as the process is repeated along wound 44. Once wound 44 is closed, suture line 13 is tied.

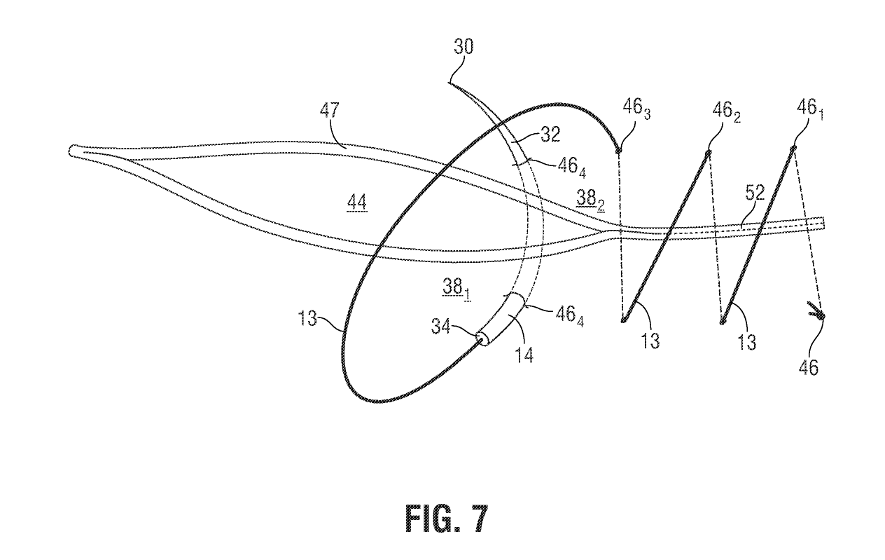

FIG. 7 illustrates aspects of bleeding suppression while closing surgical wound 44 with an embodiment of the present invention. As shown, needle 14 is inserted on one side of tissue field 38₁ of surgical wound 44 in field 48 through tissue thickness 47 that is penetrated by sharp point 30 of surgical needle 14 and, as depicted, needle 14 is pushed and/or pulled with needle holder 18 (not shown in this view) to be emergent from other side 38₂ of wound 44 as sharp point 30 penetrates thickness 47 of tissue. In a preferred embodiment and with reference to FIG. 7 and prior figures, RF energy is applied along path P from energy source 12 along conductive line 16₁ through needle holder 18 to uninsulated needle 14 and, in particular, its body 32 while in contact with tissue fields 38₁ and 38₂ thus cauterizing needle tracts 46 as created through which needle 14 is then pulled along with suture line 13. Suture line 13 is tightened to incrementally close wound 44 as shown by reference 52. This technique is conducted along wound 44 until closure along wound 44 is achieved.

Functional upper eyelid blepharoplasty is performed to restore vision blocked by excess skin, muscle and fat which typically lays across the eyelashes resulting in an impeded view by the patient. Cosmetic blepharoplasty improves patient appearance but typically has no impact on vision although functional blepharoplasty can improve both vision as well as appearance.

As those of skill will appreciate, blepharoplasty results can, in part, be dependent upon the course of healing. Improved healing and minimized scarring can be obtained by minimization of bruising which can arise from bleeding, amongst other causes. Blepharoplasty implicates small dimensions and delicate tissue and, therefore, the bleeding and concomitant bruising and occasional scarring which arises from surgical needle tracts 46 can be a substantial factor in healing and outcomes. Orbital hemorrhage can be particularly adverse and steps taken to reduce bleeding should be part of the procedure noting that skin flexibility should be maintained and scarring minimized to assist in avoidance of dry eyes and pain that can also come from excessive removal of skin. Consequently, bleeding minimization through cauterization is of value in blepharoplasty.

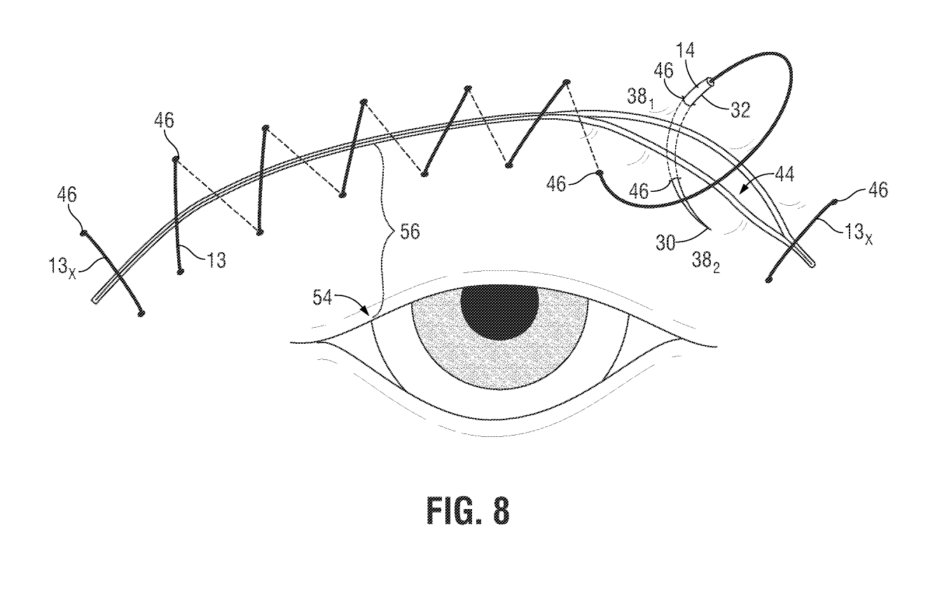

FIG. 8 depicts an embodiment of the present invention in an upper eyelid blepharoplasty to reduce bleeding. As shown, an eyelid incision wound 44 has been placed approximately 6-7 mm above eyelid margin 54 as indicated by wound height reference 56. Such incisions are preferably limited to thin eyelid skin and orbicularis muscle avoiding the thicker skin and levator muscle. Upon closure, suture is placed at the ends of wound 44 as indicated by references 13X. A running suturing technique is used to close the remainder of wound 44. The sharp point 30 of surgical needle 14 is used to puncture into a first side of tissue field 38₁ above wound 44 thus creating a needle tract 46. Sharp point 30 of surgical needle 14 is pushed up out of the tissue field 38₂ on the other side of wound 44. When the sharp point 30 of needle 14 has punctured tissue and the body of needle 14 is engaged in tissue, energy source 12 is enabled thus energizing needle 14 from which energy emanates to cauterize the respective needle tract 46. The practitioner may choose to energize the needle 14 when it is in a needle tract 46 on only one side of wound 44 although typically energization will occur when body 32 of needle 14 is engaged in tissue on both sides of wound 44.

As those of skill will recognize, cauterization seals off blood vessels and thus staunches bleeding. Needle 14 and suture line 13 are drawn through cauterized needle tracts and the suture line is pulled taut to incrementally close wound 44. As needle 14 and particularly its body 32 is within tissue of the surgical field, energy source 12 is activated by the practitioner to cauterize the tissue of the respective needle tracts and, as previously described, preferably under control of a switch controllable by the practitioner such as, for example, hand-controlled switch 24 such as a thumb switch or a foot switch 22 as depicted in earlier FIG. 1. Intermittently applied RF energy cauterizes needle tracts 46 thus minimizing bleeding.

We claim:

1. A method for closing a surgical wound comprising the steps of:
providing a surgical needle having a sharp point and to which surgical needle a suture line is attached, a needle holder, and an energy source configured for selective enablement;
providing a connective line configured to connect the energy source to the needle holder;
grasping the surgical needle with the needle holder;
puncturing with the sharp point of the surgical needle, tissue on a first side of a surgical wound thus creating a first needle tract; and
enabling the energy source so as to energize the surgical needle and thereby cauterize the first needle tract.

2. The method of claim 1 further comprising the step of puncturing with the sharp point of the surgical needle, tissue on a second side of the surgical wound thus creating a second needle tract and with the surgical needle residing in the first and second needle tracts, enabling the energy source so as to energize the surgical needle grasped by the needle holder and thereby cauterize the first and second needle tracts.

3. The method of claim 1 in which the surgical wound is coincident with upper eyelid blepharoplasty.

4. The method of claim 2 in which the surgical wound is coincident with upper eyelid blepharoplasty.

5. The method of claim 1 further comprising pulling the suture line through the cauterized tissue.

6. The method of claim 2 further comprising pulling the suture line through the cauterized tissue.

7. The method of claim 1 in which the energy source provides radio frequency (RF) energy.

8. The method of claim 7 in which the energy source provides monopolar RF energy.

9. The method of claim 1 in which the needle holder is configured with electrically insulated arms.

10. The method of claim 1 in which the needle holder is a forceps.

11. The method of claim 1 in which the energy source is controlled by a practitioner-controlled switch.

12. A method of suturing a surgical wound comprising the step of;
applying RF energy to a surgical needle to cauterize and thereby staunch bleeding from a first needle tract created by the needle's passage into tissue being sutured.

13. A system for tissue cauterization during surgery, the system comprising:
a surgical needle having a sharp point and an uninsulated body and to which surgical needle a suture line is connected;
a needle holder configured to conductively grasp the surgical needle;
a selectively operable energy source; and
a conductive line configured to provide at least a part of a conductive path between the needle holder and the selectively operable energy source, the system being configured so that when the surgical needle is conductively grasped by the surgical needle, tissue is cauterized when the selectively operable energy source is enabled.

14. The system of claim 13 in which the selectively operable energy source is configured to provide monopolar RF energy.

15. The system of claim 13 in which the system further comprises a switch which when closed enables the selectively operable energy source.

16. The system of claim 13 in which the monopolar RF energy provided by the selectively operable energy source is devised for cauterization rather than cutting of tissue.

17. The system of claim 13 in which the system further comprises a conductive contact configured for conductively contacting a patient.

18. The system of claim 13 in which the system is configured so that the energy provided by the selectively operable energy source, when said energy source is enabled, provides energy configured to flow through the conductive line and the needle holder which energy further flows into the surgical needle when the surgical needle is conductively grasped by the needle holder.

19. The system of claim 18 in which the system is configured so that the energy provided by the selectively operable energy source, when said energy source is enabled, provides energy configured to flow through the conductive line and the needle holder which energy further flows into the surgical needle when the surgical needle is conductively grasped by the needle holder and into tissue within which the surgical needle resides.

20. The system of claim 19 in which the energy provided by the selectively operable energy source is configured to cauterize tissue within which the surgical needle resides.

21. The system of claim 20 in which the energy provided by the selectively operable energy source is configured to cauterize needle tracts resulting from surgical wound closure.
